(19) United States
(12) Patent Application Publication
(10) Pub. No.: US 2023/0241163 A1
(43) Pub. Date: Aug. 3, 2023

(54) Recombinant Human CC10 Protein for Treatment of Influenza, Ebola and Coronavirus

(71) Applicant: APC RESEARCH ASSETS LLC, Rockville, MD (US)

(72) Inventor: Aprile L. PILON-CLAYTON, Rockville, MD (US)

(21) Appl. No.: 18/008,067

(22) PCT Filed: Jun. 2, 2021
(86) PCT No.: PCT/US2021/035521

§ 371 Date: Dec. 2, 2022

(51) Int. Cl. A61K 38/17; A61P 31/14

(52) U.S. Cl. CPC A61K 38/1709; A61P 31/14

(57) ABSTRACT

Methods of using recombinant human CC10 (rhCC10), also known as recombinant human uteroglobin and secretoglobin 1A1 (SCGB1A1), to reduce virus titers in the tissues of patients, particularly influenza, ebola, and coronavirus titers in lung tissues are provided. RhCC10 may be used as a therapeutic in the treatment, cure, or prevention of viral infection, particularly influenza, ebola, and coronavirus infection. More particularly, methods, including broadly the critical dosage ranges of rhCC10, intravenous and intranasal route of administration, which may be administered to treat, cure or prevent influenza, ebola, and coronavirus infection are provided. Further provided are compositions useful in the foregoing methods and in administering rhCC10 to humans.

### CROSS REFERENCE TO RELATED APPLICATIONS

This application claims benefit of a U.S. Provisional Application No. 63/033,774, the disclosure of which is incorporated herein by reference in its entirety.

### FIELD OF THE INVENTION

Embodiments of the present invention relate to methods of reducing viral titers in vivo and treating a viral respiratory or hemorrhagic infection in a patient. Embodiments of the present invention also relate to methods of treating influenza infection, including Type A influenza, particularly H1N1 influenza, ebola virus disease (EVD) also known as ebola hemorrhagic fever (EHF), and coronavirus infection. Furthermore, embodiments of the present invention also relate to methods of treating the above using intranasally-administered and/or intravenously-administered, and/or inhaled recombinant human CC10. The rhCC10 may be given alone or in combination with anti-viral drugs or other therapeutic agents that interrupt the viral replication cycle.

### BACKGROUND

Clara Cell “10 kDa” protein (CC10) or uteroglobin (UG) is a small, homodimeric secretory protein produced by several mucosal epithelia and other organs of epithelial origin (Mukherjee, 1999). CC10 consists of two identical subunits of 70 amino acid residues, each with the “four helical bundle” secondary structure motif, joined in antiparallel orientation by two disulfide bonds between Cys 3 and 69′, 3′ and 69 (Matthews, 1994; Morize, 1997). CC10 is the first member of an emerging family of small globular proteins that share the same secondary, tertiary and quaternary structure and are thought to mediate similar functions. The homodimer containing two disulfide bonds appears to be its primary form. In humans, the lung is the main site of CC10 production, while several other organs synthesize smaller amounts of mRNA encoding this protein (Singh, 1987; Sandmoller, 1994). CC10 is an anti-inflammatory and immunomodulatory protein that has been characterized with respect to various interactions with other proteins, receptors and cell types (reviewed in Mukherjee, 1999 and Pilon, 2000). Lower levels of CC10 protein or mRNA have been found in various tissue and fluid samples for a number of clinical conditions characterized by some degree of inflammation including pneumonia (Nomori, 1995).

The physiology of CC10 protein in different types of pulmonary infections has been studied in one strain of CC10 knockout mouse. In two studies in which CC10 knockout and wild type mice were each infected with either Pseudomonas aeruginosa or adenovirus, two common human respiratory pathogens, the wild type mice experienced more rapid clearing of the pathogens, with greater killing of the pathogens by the innate immune system, suggesting a benefit to CC10 deficiency during viral and bacterial infection (Hayashida, 1999; Harrod, 1998). This is consistent with earlier observations in which CC10 was reported to be an immunosuppressive agent (Dierynck, 1995; 1996), indicating that CC10 would suppress the natural immune response to an infection, whether bacterial or viral, including influenza. Thus, the administration of CC10 in the presence of a viral or bacterial respiratory infection could not be expected to benefit the patient. Subsequently, it was reported that restoration of CC10 function using recombinant human CC10 protein (rhCC10) prior to infection with respiratory syncytial virus (RSV), enabled a more rapid clearance of the infection than in untreated knockout mice (Wang, 2003). However, a recent study showed that rhCC10 can prevent the development of acquired immunity, specifically antigen-specific T cells, when present at the same time that dendritic cells are exposed to antigen (Johansson, 2007), which again indicates that administration of rhCC10 may not benefit a patient with an infection. Thus, the current state of knowledge regarding the potential hazards or benefits of rhCC10 treatment during a respiratory infection is conflicting and allows no conclusions to be drawn regarding the safe and/or efficacious use of CC10 to treat different types of respiratory infections. No information regarding the effect of CC10 on influenza infection is available. We report herein, direct verification of the efficacy of rhCC10 against influenza Type A in vivo, direct verification of the anti-viral effects of CC10 at the cellular level, its mechanism of action, and its potential use to treat and/or prevent viral infection, and, in particular, influenza infection.

Influenza has caused four major outbreaks (1889, 1918, 1957, and 1968) in the past 120 years, causing the deaths of an estimated 50-100 million people worldwide. Influenza is an orthomyxovirus, an RNA virus that is transmitted by aerosols as well as by direct contact of contaminated surfaces with nasal mucosa and targets respiratory epithelial cells. Influenza infection may cause severe symptoms, including fever, sore throat and muscle aches, malaise, weight loss, respiratory congestion, and sometimes respiratory failure and death. Influenza elicits an acquired immune response (cytotoxic T cells and antibodies) that typically clears the infection in 1-2 weeks in normal healthy individuals. Several subtypes of influenza that infect humans, including avian influenza (H5N1), seasonal influenza H3N2, and swine flu (H1N1), can be treated with antiviral agents such as neuraminidase inhibitors. However, the rapid rate of mutation in influenza has led to the development of drug-resistant strains (Moscona, 2009), such that widespread use of antiviral agents for prevention and or treatment will lead to acceleration of the development of resistance to these drugs. New therapeutic agents are therefore needed to treat, cure and prevent influenza infection. Likewise, there are no approved therapies for the vast majority of viral infections in the respiratory tract and other body systems.

Ebola is a hemorrhagic fever virus, also known as a filovirus, originating in Africa with a very high mortality rate. It is highly contagious and is transmitted by contact with bodily fluids from an infected person or animal. It attacks the endothelial cells of blood vessels, breaking down blood vessels, allowing blood and serum to leak from the circulatory system. Death often occurs with a few days of the appearance of hemorrhagic symptoms. There is currently a widespread epidemic of ebola in West Africa and no proven anti-viral medication or vaccine available. New therapeutic agents are therefore, needed to treat, cure, and prevent ebola infection.

Coronaviruses are widespread viruses, divided into Alpha and Beta type families, which infect the respiratory and gastro-intestinal tracts in many species of mammals, including humans. Several strains of coronaviruses infect humans and cause only mild cold-like and/or gastro-intestinal symptoms, while others cause very severe, sometimes lethal infections in humans. In the past two decades, 3 deadly coronaviruses from the Beta type family, have emerged, including the Severe Acute Respiratory Syndrome coronavirus (SARS-CoV1) outbreak in 2003, the Middle Eastern Respiratory Syndrome coronavirus (MERS-CoV) outbreak in 2017-2018, and the COVID-19 coronavirus (also called SARS-CoV2) outbreak spreading across the globe in 2019-2020. There are currently no approved anti-viral medications to treat these coronaviruses and new therapeutics are therefore, needed to treat, cure, and prevent severe coronavirus infection.

Heparan sulfate proteoglycans (HSPGs) are expressed on the outer membranes of cells and have carbohydrate side chains that together make up the glycocalyx, which is a gelatinous protective layer that regulates penetration of fluids, ions, nutrients, signaling ligands, and other molecules into and between cells. Another name for HSPG carbohydrate side chains is glycosaminoglycans (GAGs), including heparan sulfate, chondroitin sulfate, and their derivatives. Syndecans and glypicans are the two main families of HSPGs. Syndecans have a transmembrane region that connects to the cytoskeletal matrix, facilitating formation of clathrin-coated pits and endosomes for transport of receptor-ligand complexes and uptake of other materials inside the cell. Syndecans may act as co-receptors to facilitate cell signaling by various ligands, such as growth factors, by loosely aggregating the ligands in their GAGs and bringing or holding them in closer proximity to their specific receptors. In contrast, glypicans do not span the membrane but are instead anchored in the membrane by glycerophosphoinositol and are linked to the signal transduction apparatus of the plasma membrane and are involved in clathrin-independent endocytosis of extracellular materials via cholesterol-rich lipid rafts. GAGs found in HSPGs include heparate sulfate and chondroitin sulfate, with minor carbohydrate variants present in different tissues. The degree of sulfation of GAGs is critical to their binding properties and thought to be an important regulator of homeostasis. Another major component of the glycocalyx of mammalian cells is hyaluronic acid (HA), which is not attached to any membrane-bound protein. All cells are normally surrounded by a glycocalyx, which may range in thickness from 20-300 nm.

HSPGs in cell membranes and glycocalyxes of different tissues are often involved in initiation of bacterial, fungal, viral, mycobacterial, parasitic, and other types of infections by facilitating receptor binding, cellular adhesion, and/or cellular uptake of the infectious agent. RhCC10 and other SCGB preparations binding to HSPGs block the interactions of many infectious agents, including viruses, with their cellular receptors, including HSPGs, and/or cellular uptake that requires syndecan function.

### OBJECTS OF THE INVENTION

The foregoing provides a non-exclusive list of the objectives achieved by embodiments the present invention:

It is an object of embodiments of the invention to reduce pulmonary viral titer and thereby treat, cure or prevent influenza infection, especially Type A influenza infection, and more especially strain H1N1 influenza infection.

It is a further object of embodiments of the invention to reduce pulmonary viral titer and treat, cure, or prevent influenza infection by administering CC10 by the intravenous route, the inhaled route, or the intranasal route (according to PCT 2009), or by a combination of routes.

It is another object of embodiments of the invention to reduce viral titer and thereby treat, cure or prevent ebola viral infection.

It is a further object of embodiments of the invention to reduce viral titer and treat, cure, or prevent ebola viral infection by administering CC10 by the intravenous route, the inhaled route, or the intranasal route (according to PCT 2009), or by a combination of routes.

It is another object of embodiments of the invention to reduce viral titer and thereby treat, cure or prevent coronavirus viral infection.

It is a further object of embodiments of the invention to reduce viral titer and treat, cure, or prevent coronavirus viral infection by administering CC10 by the intravenous route, the inhaled route, or the intranasal route (according to PCT 2009), or by a combination of routes.

It is another object of embodiments of the invention to reduce viral titer and treat, cure, or prevent viral infection by administering CC10 by the intravenous route, the inhaled route, or the intranasal route, the oral route, the intravaginal route, or by a combination of routes.

It is yet another object of embodiments of the invention to inhibit viral replication at the cellular level using CC10 or other members of the secretoglobin family.

### SUMMARY OF THE INVENTION

These and other objects, features and advantages are achieved by embodiments of the invention by administering rhCC10 in a dosage range given at appropriate intervals, or in one dose, to reduce viral titer and treat, cure or prevent viral infection.

These and other objects, features and advantages are also achieved by embodiments of the invention by administering CC10 in a dosage range given at appropriate intervals or in one dose where a patient is diagnosed with a viral infection by symptoms characteristic of the particular virus, and/or by detection of virus in patient samples through culturing of the virus, immunological detection of the virus, and/or detection of the viral nucleic acid, using standard methods.

These and other objects, features and advantages are also achieved by embodiments of the invention by administering CC10 in a dosage range given at appropriate intervals or in one dose where a patient is diagnosed with an influenza infection by one or more symptoms of fever, myalgia, and congestion, and/or by detection of influenza virus in patient samples (nasal lavages, blood or sputum samples) through culturing of the virus, immunological detection of the virus, and/or detection of the viral nucleic acid, using standard methods.

These and other objects, features and advantages are also achieved by embodiments of the invention by administering CC10 in a dosage range given at appropriate intervals or in one dose where a patient is diagnosed with an ebola viral infection by one or more symptoms of fever, sore throat, muscle pain, and headaches, vomiting, diarrhea, rash, decreased function of the liver and decreased function of the kidneys and/or by detection of ebola virus in patient samples (nasal lavages, blood or sputum samples) through culturing of the virus, immunological detection of the virus, and/or detection of the viral nucleic acid, using standard methods.

These and other objects, features and advantages are also achieved by embodiments of the invention by administering CC10 in a dosage range given at appropriate intervals or in one dose where a patient is diagnosed with coronavirus infection by one or more symptoms of fever, sore throat, muscle pain, and headaches, vomiting, diarrhea, chest imaging (ground glass opacities), hypoxia, dyspnea, decreased function of the liver and decreased function of the kidneys and/or by detection of coronavirus in patient samples (nasal lavages, blood or sputum samples) through culturing of the virus, immunological detection of the virus, and/or detection of the viral nucleic acid, using standard methods.

In certain aspects of the invention, CC10 is administered intranasally in a dose divided about equally between each nostril in a range of 1.5 micrograms to 1.5 milligrams per kilogram of body weight per day, or in multiple doses which taken together achieve this dosage range on a daily basis to reduce pulmonary viral titer and treat, cure or prevent influenza, ebola, or coronavirus infection.

In another aspect, CC10 is administered intravenously in a dose of up to 20 milligrams per kilogram of body weight per day, or in multiple doses which taken together achieve this dosage range on a daily basis to treat, cure or prevent influenza, ebola or coronavirus infection.

In another aspect, a non-human CC10 protein is administered in a dosage range given at appropriate intervals or in one dose where a patient is diagnosed with a viral infection by symptoms characteristic of the particular virus, and/or by detection of virus in patient samples through culturing of the virus, immunological detection of the virus, and/or detection of the viral nucleic acid, using standard methods.

In yet another aspect, another member of the CC10 family of proteins, collectively known as secretoglobins, is administered in a dosage range given at appropriate intervals or in one dose where a patient is diagnosed with a viral infection by symptoms characteristic of the particular virus, and/or by detection of virus in patient samples through culturing of the virus, immunological detection of the virus, and/or detection of the viral nucleic acid, using standard methods.

### BRIEF DESCRIPTION OF THE DRAWINGS

FIG. 1 is a bar graph depicting H1N1 viral load at 2 days in lungs of infected cotton rats treated with intranasal rhCC10. Viral titer is expressed as (x 107) TCID50/gram of tissue.

FIG. 2 is a bar graph depicting H1N1 viral load at 2 days in lungs of cotton rats treated with intraperitoneal injection of rhCC10. Viral titer is expressed as (x 107) TCID50/gram of tissue.

FIG. 3 is a bar graph depicting inhibition of viral replication in cultured cells by rhCC10. RhCC10 was added to the culture medium of HEp2 cells at 100 micrograms/ml, 300 micrograms/ml, and 1 milligram/ml and left for 4 hours. Then the medium was removed and replaced and cells were infected with RSV for 1 hour. The cells were then washed to remove excess virus and rhCC10 was added back and incubated for another hour. Then cells were washed to remove excess CC10. Viral titers in culture medium were measured at 4 days post-infection. Each CC10 concentration was performed in triplicate.

FIG. 4 is a bar graph comparison of rhCC10 antiviral effects when given pre-infection and post-infection. HEp2 cells were treated with 1 mg/ml rhCC10 and infected with RSV as in FIG. 3. In addition, 1 mg/ml of rhCC10 was given at one hour after infection (treatment D0), 24 hours after infection (treatment D1), and 48 hours after infection (treatment D2). Viral titers in culture media were measured on day 4 post-infection.

FIG. 5 shows an agarose gel electrophoretic mobility shift assay (EMSA) demonstrates non- covalently linked complexes between recombinant human SCGBs and LPS. Panels A and B show complexes between two different batches of rhSCGB1A1 and 3 types of LPS, including 0111:B4, O55:B5, and EH100.

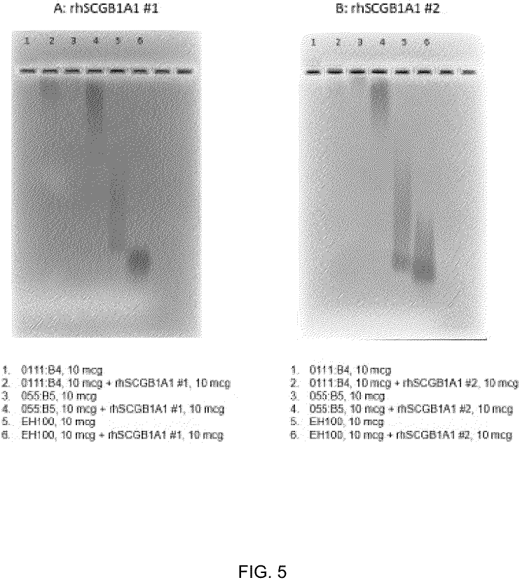

FIG. 6 shows an agarose gel EMSA demonstrates non-covalently linked 3-way complexes between rhSCGB1A1 and LPS EH100 and heparin. The order in which each component was added is indicated by the order in which the components are listed for each lane.

FIG. 7 depicts Far-Western blots illustrating binding of rhSCGB1A1 to recombinant human SDC1, SDC4, and GPC3 in the presence of two different types of LPS (EH100 and O55:B5). The SCGB1A1 bound to the HSPGs were detected with 3 different anti-human SCGB1A1 antibodies, including a rabbit polyclonal (APC-012), a goat polyclonal (APC-013), and a mouse monoclonal (E11). BSA was used as a negative control and rhSCGB1A1 was spotted onto the blots as a positive control. Panel A shows rhSCGB1A1 binding to recombinant human SDC1 and GPC3 in the presence of 2 different types of bacterial endotoxin. Panel B shows rhSCGB1A1 and rhSCGB3A2 binding to hSDC1, hSDC4, and hGPC3 in the presence and absence of LPS (EH100). Panel C shows rhSCGB1A1 and ROS-modified rhSCGB1A1 binding to hSDC1, hSDC4, and hGPC3 in the presence and absence of LPS (EH100).

FIG. 7A is a summary schematic of rhCC10 expression in bacteria and recovery.

FIG. 8 is a schematic overview of an rhCC10 purification process.

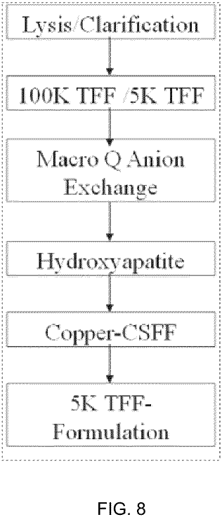

### DETAILED DESCRIPTION

Embodiments of the present invention relate to the use of CC10 to reduce pulmonary viral titer and treat, cure or prevent influenza infection. The CC10 is preferably a recombinant human CC10 protein (rhCC10) obtained by the processes described in U.S.

Pat. App. Publication No.: 20030207795 and PCT/US09/43613 attached hereto at Exs. A and B respectively, all of which are incorporated by reference in their entirety, or via any other process which yields pharmaceutical grade rhCC10. The rhCC10 of the embodiments of the present invention may be administered with, without, before or after other intranasal, pulmonary, or systemic therapy.

Without limiting the scope of possible synthetic processes that may be used to make human CC10, the recombinant human CC10 (aka uteroglobin) that is active in suppressing viral replication in vitro and in vivo was synthesized and characterized as described in U.S. Pat. App. Publication No.: 20030207795.

Preparations of rhCC10 for intranasal administration as described in PCT/US09/43613 represent further embodiments of the present invention that may be used to suppress viral replication in vivo, particularly in the nasal passages and sinuses.

### Dosages

Preferably, in treating or preventing influenza infection, rhCC10 is administered intranasally, to each nostril 1-3 times per day, for 7-14 days, and every other day thereafter for another 14 days, and thereafter as needed. More preferably, rhCC10 is administered as soon as the patient begins to experience fever, myalgia, and congestion or is diagnosed with influenza. In particular, higher doses given more frequently, or as a continuous infusion, are effective in controlling viral replication to treat more severe infections. More specifically, discrete doses of 0.5, 1.5, 5.0, and 10.0 mg/kg may be given intravenously or by another route of administration, daily for up to 14 days, or given up to 4 times per day (every 6 hours) for up to 7 days, in order to treat the infection.

The rhCC10 may be produced in a process comprising the steps of: a) providing a bacterial expression system capable of expressing rhCC10; b) inoculating a fermenter with an inoculum comprising the bacterial expression system to form a fermentation culture; c) adding an induction agent to the fermentation culture to induce the expression of rhCC10 by the bacterial expression system; d) harvesting the rhCC10 expressed in step c; and e) purifying the rhCC10 harvested in step d, wherein the purifying step comprises the use of at least one filter and at least one ion exchange column, as described in U.S. Pat. App. Publication No.: 20030207795. The rhCC10 may also be expressed in alternative bacterial, fungal, insect, mammalian, or plant expression systems and purified to meet specifications for a pharmaceutical product suitable for administration to humans using standard methods.

Specifications and testing results for pharmaceutical grade rhCC10, according to U.S. Pat. App. Publication No.: 20030207795, that may be used to reduce viral titers include the following:

| | |
|---|---|
| Test | Specification |
| Color | Clear, colorless |
| Appearance | No turbidity |
| Homogeneity | Homogeneous |
| Purity | ❑ 95% |
| Aggregation | ❑ 5% |
| Sterility | Sterile |
| Biological activity | Positive |
| Bacterial nucleic acid | <100 pg/dose |
| Mass spectroscopy | App 16110 |
| pH | 5-8 |
| Isoelectric focusing | 4.7 +/- 1 |
| Free Thiol | <10% (w/w) |
| LAL | <5 EU/mg |
| Copper | <16 ❑M |

In a further embodiment, the rhCC10 of the present invention that inhibits viral replication also inhibits phospholipase A₂ (PLA₂) enzymes, as described in U.S. Pat. App. Publication No.: 20030207795.

To effectuate the desired outcomes which are further described below, reference is made to methods of administration described in the following embodiments:

In one embodiment, a dose or multiple doses of intranasal rhCC10 equaling a dose ranging from about 1.5 micrograms to about 5 milligrams per kilogram of body weight per day may be administered. In another embodiment, rhCC10 may be administered in the dose range on a daily basis. In yet another embodiment, rhCC10 may be administered in the dose range on a daily basis for at least seven days consecutively. In still a further embodiment, rhCC10 may be administered in the dose range on a daily basis for at least 14 days consecutively. In still another embodiment, rhCC10 may be administered in the dose range every other day for 30 days consecutively. In yet another embodiment, rhCC10 may be administered in tapered dosages daily for ten consecutive days, said tapered dosages comprising a high dose at each administration for the first three days, an intermediate dose at each administration for the second three days, and a low dose at each administration for the last four days. In yet still another embodiment, rhCC10 may be administered in the dose range or in tapered doses up to three times per day, approximately every eight hours.

In another embodiment, the above doses of rhCC10 may be administered intranasally to the patient as an aerosol, by intranasal spray or lavage, or by deposition of a gel or cream, or other method of instillation in the nasal passages.

In another embodiment, the above doses of rhCC10 may be administered by inhalation to the patient as an aerosol, by nebulizer or metered dose inhaler, or other method of direct application to the lungs and airways.

In another embodiment, in treating or preventing influenza infection, rhCC10 is administered intravenously, in doses of 15 micrograms to 20 milligrams per kilogram of body weight, 1-3 times per day, for 7-14 days, and every other day thereafter for another 14 days, and thereafter as needed. In yet another embodiment, rhCC10 may be administered in tapered dosages daily for ten consecutive days, said tapered dosages comprising a high dose at each administration for the first three days, an intermediate dose at each administration for the second three days, and a low dose at each administration for the last four days. In yet still another embodiment, rhCC10 may be administered in the dose range or in tapered doses up to three times per day, approximately every eight hours.

In another embodiment the above doses of rhCC10 may be administered to the patient using a combination of intranasal, inhaled, and intravenous routes. In a further embodiment, rhCC10, in accordance with the methods described above, may be administered prior to, during or after anti-viral therapy, anti-biotic therapy, decongestant, anti-histamine, mucolytic, expectorant, mucus suppressor, surfactant, bronchodilator, vasoconstrictor, sinus pain analgesic, or other typical therapy. In still another embodiment, rhCC10, in accordance with the methods described above, may be administered to reduce pulmonary viral titer and treat, cure, or prevent influenza infection. In still another embodiment, rhCC10 may be combined with an antiviral or other drug before the combination is administered to a patient infected with influenza, ebola, coronavirus, or any other type of virus.

The doses of rhCC10 and application methods described above may be administered daily, more than once daily, three times daily, every other day or in a tapered fashion depending upon the severity of influenza infection being treated, the patient’s overall health, and whether underlying conditions are present. For example, the more severe the infection, the higher the amount of rhCC10 would be required to effectively treat it. It is understood that a physician would be able to monitor and adjust doses, formulations, and application methods as needed based on the patient’s symptoms and responses to therapy and within the parameters and dose ranges described in the embodiments of the present invention.

### Formulations

Intranasal formulations, devices, and methods by which rhCC10 may be administered intranasally have been described in PCT/US09/43613, which is incorporated herein by reference in its entirety. The intravenous formulation of rhCC10 consists of a 5.5 mg/ml solution in 0.9% saline and has been described in U.S. Pat. App. Publication No.: 20030207795, which is incorporated herein by reference in its entirety.

### Example 1

### Propagation and Titer Determination of Influenza Virus

The A/PR/8/34 Influenza A viral strain (H1N1), purchased from the American Type Culture Collection (Manasass, Virginia, USA) is prepared. Influenza virus is propagated in MDCK cells (ATCC catalog# CCL-34) by infecting 60% confluent cell monolayer (150 cm² flasks) with flu virus at a multiplicity of infection (MOI) of 0.01. Three to four days later, when cytopathic effect is generalized and most of the cells have detached from the culture vessel, the cells and supernatants are harvested. Cells are removed by centrifugation (800 g) and the supernatant filtered (0.45 µm) and centrifuged (18000 g) for 2 hours at 4° C. to pellet viruses. The viral pellet is resuspended in DMEM medium, aliquoted and stored at -150° C. Influenza virus titer is determined by applying 0.1 mL of serially diluted viral stocks to MDCK cell monolayers in a 96-well plate cultured in the presence of 0.1% bovine serum albumin and trypsin. Three days later, cytopathic effects were scored and the tissue culture infectious dose 50% (TCID₅₀) is determined using Kärber’s method.

Processing of Lung Tissues for viral load analysis. Sections of the left and right lobes from lungs of infected mice and cotton rats are aseptically removed, weighed and homogenized in 1 ml of DMEM medium for 45 seconds using a tissue tearor apparatus (model#985-370, Biopspec Products Inc.) at a setting of 5. Homogenates are centrifuged at 3000 g for 20 minutes. Clarified supernatants are collected, and stored frozen at -150° C. until used. Determination of Viral Titers. Viral titers in amplified viral stocks and in lung homogenates were determined by serial dilution followed by plaque forming assay (PFA) or foci forming assay (FFA). Plaque forming units (PFU) and foci forming units (FFU) per milliliter of original sample were calculated prior to the start of the study. One set of influenza samples sufficient for carrying out PFA and FFA was stored and PFU and FFU were determined after the completion of the studies. Serial dilutions of cultured virus in clarified media (DMEM with 1% BSA) were prepared across 10¹ to 10⁸ dilution range. Each dilution is evaluated by a plaque forming assay (PFA) and a foci forming assay (FFA). Culture titers typically yield 10⁷ - 10⁹ pfu/ml for influenza.

### Example 2

### Intranasal Administration of rhCC10 to Reduce Pulmonary Influenza Virus Titer

The cotton rat (S. hispidus), a type of vole, is an animal model in which influenza replicates and generates a mild respiratory infection (Ottolini, 2005). The animals are infected by intranasal inoculation with influenza virus and pulmonary viral titers peak two days (about 48 hours) after inoculation. This model is used to screen for compounds that inhibit influenza replication in vivo.

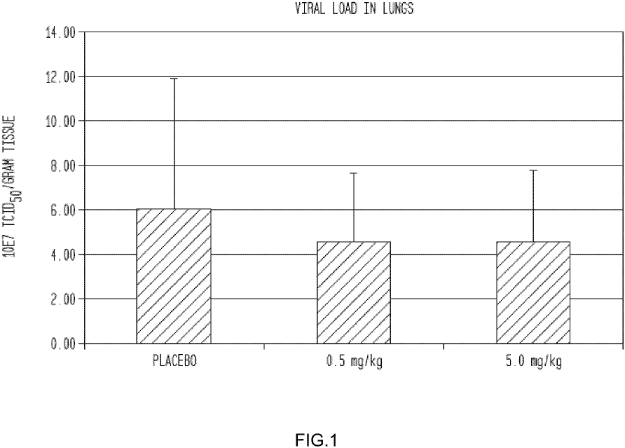

Pathogen free cotton rats were purchased from Virion Systems, Inc. (Rockville, MD). A total of eighteen cotton rats (S.hispidus, 6-8 weeks old) were infected with Type A influenza (A/PR/8/34), strain H1N1, by intranasal inoculation using 10⁷ TCID₅₀ in 0.1 ml volume for each rat. Six animals received a placebo (0.9% NaC1), six animals received 0.5 mg/kg of rhCC10 and six animals received 5.0 mg/kg of rhCC10 by intranasal instillation 2 hours before viral inoculation. Animals were sacrificed on day 2 post-infection when viral titers are typically highest and viral load was determined in lung tissue. FIG. 1 illustrates the reductions in viral titer in lung tissue that were observed in the both rhCC10 dose groups. Viral titer in lung is expressed as (x 10⁷) TCID₅₀/gram of tissue.

### Example 3

### Systemic Administration of rhCC10 to Reduce Pulmonary Influenza Virus Titer

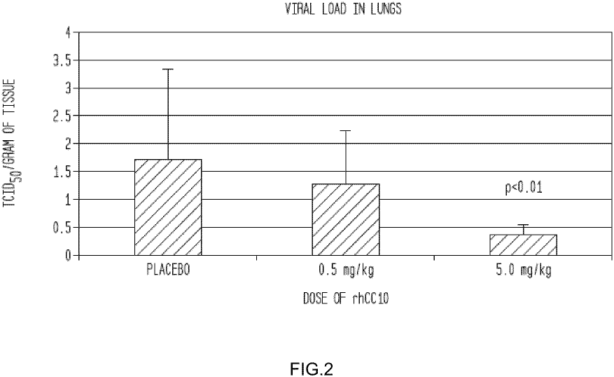

A total of eighteen cotton rats (S.hispidus, 6-8 weeks old) were infected with Type A influenza (A/PR/8/34), strain H1N1, by intranasal inoculation using 10⁷ TCID₅₀ in 0.1 ml volume for each rat. Six animals received a saline placebo, six animals received 0.5 mg/kg of rhCC10 and six animals received 5.0 mg/kg of rhCC10 by intraperitoneal injection (IP). The IP route results in significant amounts of circulating rhCC10 and simulates the intravenous route of administration in humans. Each animal received a total of six doses of either placebo or rhCC10 approximately every 12 hours, including two doses (morning and afternoon) on the day before infection, two doses on the day of infection, and two doses on the day after infection (3 doses before infection, 3 doses after infection). Animals were sacrificed on day 2 post-infection when viral titers are typically highest and viral load was determined in lung tissue. FIG. 2 illustrates the statistically significant reduction (p<0.01) in viral titer in lung tissue that was observed in the 5 mg/kg rhCC10 dose group, and the trend towards a lower viral titer in the 0.5 mg/kg dose group. Viral titer in lung is expressed as (x 10⁷) TCID₅₀/gram of tissue.

Based on the foregoing, rhCC10 has been found to reduce viral titer in a respiratory infection, indicating the use of rhCC10 to treat, cure and/or prevent influenza infection. Accordingly, embodiments of the present invention provide an intranasal, and intravenous, or a combination rhCC10 based therapy effective at treating, curing or preventing influenza infection.

### Example 4

### CC10-Mediated Inhibition of Viral Replication at the Cellular Level

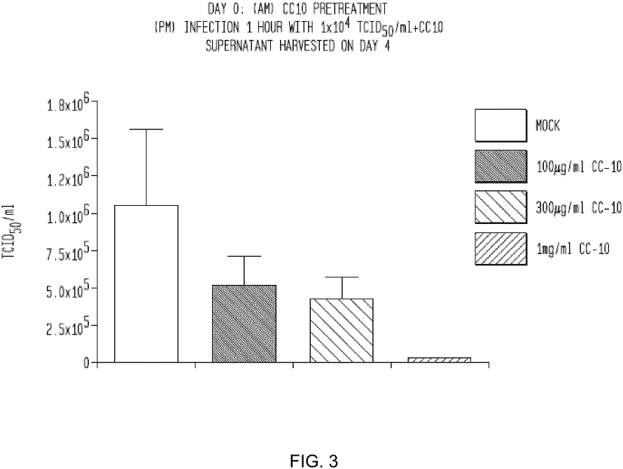

HEp2 cells (ATCC, Manassas, VA) were used to propagate RSV, strain A-2 (Advanced Biotechnologies, Inc., Columbia, MD) and generate viral stocks. Cells were plated at 50,000 cells/well in 48 well plates and grown in MEM with 10% FBS to ~80% confluence. Cells were pre-treated with CC10 in 0.5 mL MEM for 4 hours. Medium was then changed and RSV infections were performed using 1 x 106 TCID50 per 100 mm TC dish for 1 hour. Non-adsorbed virus was removed by washing and 0.5 ml of MEM with 2% FBS, 4 mM L-glutamine, and rhCC10 was added. Supernatants were collected on day 4 post infection and the virus titrated. FIG. 3 shows that a concentration of 1 mg/ml CC10 virtually eliminated RSV production, while 100 and 300 micrograms/ml showed a ~3-fold decrease.

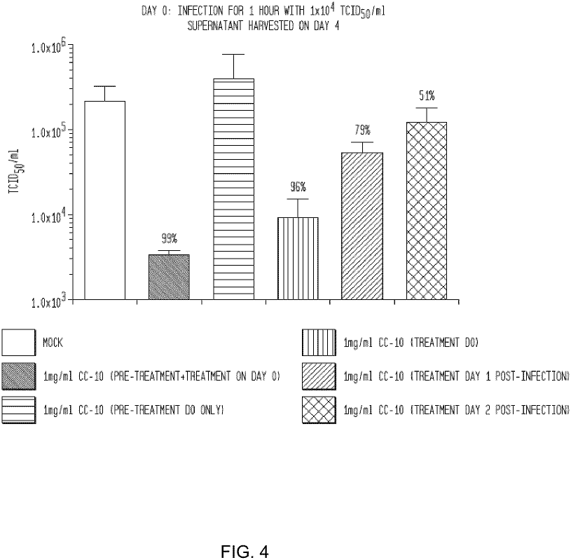

CC10 also inhibited viral replication in cells when given at 1, 24, and 48 hours after infection. FIG. 4 shows that rhCC10 is effective at reducing viral titer not only when added before infection, but also when added after infection. This is the first report of a direct anti-viral activity of CC10 at the cellular level and illustrates the potential utility of rhCC10 as an anti-viral therapy for post-exposure treatment.

### Example 5

### CC10 Anti-Viral Mechanism of Action

The phenotype of airway epithelial cells in the CC10 knockout mouse illustrates that in the absence of CC10, the distribution of intracellular organelles is abnormal, that abnormal stacked membranous structures are present, and that secretion of other proteins made by the cell is disrupted. We surmise that this phenotype means that CC10 plays an active role in transport of secretory vesicles from the Golgi apparatus to the plasma membrane of the cell. CC10 also modulates the uptake and processing of antigens in antigen-presenting cells. We interpret these observations to mean that CC10 is an important factor in the transport of materials both out of and in to many types of cells. We therefore infer that CC10 inhibits viral replication by interfering with viral transport in the cell. Since all viruses rely upon cellular transport processes to invade the cell and replicate, CC10 can be expected to inhibit the replication of all viruses. Likewise, other secretoglobins, which share similar structure to CC10, can also be expected to inhibit viral replication at the cellular level. Similarly, peptides derived from CC10 and other secretoglobins that modulate cellular transport processes can also be expected to inhibit viral replication.

### Example 6

### Inhibition of Ebola Virus Replication

Methods: Vero cells were seeded in 96-well plates and cultured to 90-100% confluence. One hour prior to infection, culture media (EMEM/NEAA, 5% FBS) was replaced with fresh media with or without rhCC10 (1.5 or 0.5 mg/ml). After one hour of exposure to rhCC10, the media was replaced with fresh media containing Ebola virus (EBOV) at a multiplicity of infection (MOI) of 3.0, 0.3, or 0 (no virus), with our without rhCC10 at the two concentrations. Virus was incubated with the cells for one hour at 37° C., then the media containing EBOV was removed, the cells were washed with PBS, the media containing rhCC10 was added. Cells were incubated at 37° C. for 24, 48, and 72 hours, after which the plates were fixed in 10% formalin to assay for plaques. To decontaminate the plates were kept in formalin for 72 hours, then placed in fresh formalin. Plates were then washed with PBS and blocked with 1% BSA overnight at 4° C. Plates were washed with PBS to remove the blocking solution and incubated in PBS containing Hoechst 33352 nuclear stain at 4° C. After 3-4 hours, the plates were incubated with a human antibody (KZ52) that recognizes the Ebola glycoprotein (EBOV GP) for 20 minutes on a platform shaker. The plates were then washed in PBS and secondary antibody (goat anti-human AlexaFluor488) was added. After 20 minutes on a platform shaker, the plates were washed and placed in PBS containing the plasma membrane dye (CellMask Deep Red) and left overnight at 4° C. The plates were imaged the next day with an Operetta system. GP-positive cells were counted and compared to total cell number to determine the percentage of cells infected with the EBOV virus.

Results: The percentage of cells infected with EBOV is the ratio of the number of EBOV GP-positive cells to the total number of Hoechst stained nuclei. At 24 hours post-infection, very few cells were infected at either MOI. At 48 hours post-infection, over 20% of the cells were infected at the lowest MOI in the absence of rhCC10. At 48 hours post-infection at the higher MOI, there was a clear dose-dependent and statistically significant (ie. p value < 0.05) reduction in the number of infected cells at both concentrations of rhCC10. At 72 hours post-infection, the reduction in the number of infected cells was significant at both MOI at the highest concentration of rhCC10 (⅕ mg/ml). The results of the experiment are shown in the Table 1 below:

TABLE 1

| | | Percent Cells Infected | | | | | |
|---|---|---|---|---|---|---|---|
| | | 24 hours | | 48 hours | | 72 hours | |
| | MOI | 0.3 | 3.0 | 0.3 | 3.0 | 0.3 | 3.0 |
| rhCC10 (mg/ml) | 0 | 1 | 15 | 26 | 66 | 43 | 53 |
| | 0.5 | 1 | 14 | 23 | 51** | 31 | 36 |
| | 1.5 | 1 | 19 | 10 | 35*** | 19* | 30** |

There were no differences in cell viability at 72 hours between cells treated with 0, 0.5, and 1.5 mg/ml rhCC10 that were not infected with EBOV, indicating that rhCC10 was not toxic to the cells. Taken together these data indicate that rhCC10 inhibits replication of the Ebola virus, thereby reducing viral titers.

### Example 7

### Binding of LPS by rhCC10

Binding of human SCGB1A1 to bacterial lipopolysaccharide (LPS), also known as endotoxin. LPS is made up of lipid, carbohydrate chains (polysaccharide), and a small amount of protein derived from bacterial cell membranes. LPS is an extremely potent inflammatory mediator and must be specifically reduced to a very low or undetectable level in medicinal preparations in order to minimize toxicity. We observed that synthetic human SCGB1A1 (rhSCGB1A1) binds to LPS using an electrophoretic mobility shift assay (EMSA) in which the migration of LPS through an agarose gel was accelerated in the presence of SCGB1A1 as shown in FIG. 1. The agarose gel EMSA of FIG. 1 demonstrates non-covalently linked complexes between recombinant human SCGB1A1 and LPS. Panels A and B show complexes between two different batches of rhSCGB1A1 and 3 types of LPS, including 0111:B4, O55:B5, and EH100. Briefly, the EMSA method used 10 micrograms (mcg) of three different preparations of LPS dissolved in ultrapure water and incubated with and without rhSCGB1A1 at 37° C. for 30 minutes. The samples were mixed with loading buffer then loaded onto a 0.8% Tris-acetate-EDTA agarose gel and electrophoresed at 100V until the dye front reached the bottom of the gel. The gel was washed in ultrapure water and immersed in 0.2 M imidazole for 20 minutes with gentle agitation. The gel was washed again in ultrapure water then incubated in 0.2 M zinc sulfate for several minutes until bands became visible. The gel was washed again in ultrapure water to stop the staining and scanned with a densitometer. The agarose gel EMSA shown on FIG. 2 demonstrates non-covalently linked 3-way complexes between rhSCGB1A1 and LPS EH100 and heparin. The order in which each component was added is indicated by the order in which the components are listed for each lane.

### Example 8

### Detection of Complex Between SCGB, LPS, and Heparin

The SCGB1A1-LPS complex also binds to carbohydrate side chains and we used the EMSA to show this. FIG. 2 shows that the addition of heparin, a low molecular weight preparation of HSPG protein side chains derived from porcine intestines and used medically as an anticoagulant, shifts the SCGB1A1-LPS complex, creating a SCGB1A1-LPS-heparin complex. This 3-component complex is formed regardless of the order in which the components are added to the mixture. This discovery demonstrates that SCGB1A1 can form a 3-way complex with LPS and a HSPG side chain and we infer that the property of forming a 3-way complex among these components is conserved among different SCGBs.

### Example 9

### Binding of SCGB1A1-LPS Complexes to HSPGs in Vitro

Recombinant human SDC1, SDC4, and glypican-3 (GPC3), both expressed in cultured human cells (HEK293) in order to best approximate human HSPG protein side chains in humans in vivo, were purchased from a commercial vendor. The binding of the rhSCGB1A1-LPS complexes to these three HSPG proteins was investigated using a “Far Western” dot blot method. Briefly, 200 ng each of SDC1, SDC4, GPC3, rhSCGB1A1 (positive control), and bovine serum albumin (BSA) (negative control), all in PBS pH 7.4, were spotted onto nitrocellulose membranes and allowed to dry. The membranes were blocked in 5% non-fat dry milk in PBS pH 7.4 for 1 hour at room temperature. The rhSCGB1A1-LPS complexes were prepared as in Example 1 (1:1 weight ratio) during the blocking step. After blocking, the membranes were equilibrated in citrate buffer, pH 6.5, then the rhSCGB1A1-LPS complexes were diluted in citrate buffer, pH 6.5 to 50 mcg/ml each of rhSCGB1A1 and LPS, added to the blocked membrane, and incubated overnight at 4° C. with gentle agitation. The membrane was washed with PBS pH 7.4, 0.1% Tween-20 (PBS-T) and incubated in the primary anti-hSCGB1A1 antibody. Three different antibodies were tested, including a rabbit polyclonal, a goat polyclonal, and a mouse monoclonal as shown in FIG. 3. Each antibody was diluted 1:1,000 in 0.1% non-fat dried milk in PBS-T and incubated with the blot for 1 hour at room temperature with gentle agitation. The membranes were washed with PBS-T and incubated in secondary antibody, which were each conjugated to alkaline phosphatase enzyme, diluted 1:8,000 in 0.1% non-fat dried milk in PBS-T, and incubated with the membranes for 1 hour at room temperature. The membranes were washed with PBS-T then incubated in NBT/BCIP to develop color. FIG. 3A shows the first result of these experiments. All three antibodies showed rhSCGB1A1-LPS complexes bound to both SDC1 and GPC3. However, the reaction was much stronger for LPS EH100 than for O55:B5 and for GPC3 compared to SDC1, with the rabbit polyclonal antibody providing the strongest signal. Thus, the 3 component complexes are formed regardless of which type of LPS and which HSPG protein was used. This discovery demonstrates that SCGB1A1 also forms a 3-way complex with LPS and a HSPG protein and we infer that the property of forming a 3-way complex among these components is conserved among different SCGBs. FIG. 3B shows the second set of results for these experiments in which the same conditions were used as in FIG. 3A, except that the binding of both hSCGB1A1 and hSCGB3A2 to the three HSPGs, with and without LPS (EH100) present was examined. Both SCGBs bind to all three HSPGs with and without LPS present, although hSCGB1A1 binding to SDC1 and GPC3 is barely detectable in this particular set of blots. Both hSCGB1A1 and hSCGB3A2 bind to SDC4 more strongly than they bind to GPC3 or SDC1, however, hSCGB1A1 binds more strongly to GPC3 than to SDC1 while hSCGB3A2 binds more strongly to SDC1 than to GPC3. FIG. 3C shows the third set of results for these experiments in which the same conditions were used as in FIG. 3B. A total of five different preparations of hSCGB1A1, including unmodified hSCGB1A1 (non-reduced hSCGB1A1) and four preparations of hSCGB1A1 chemically modified by exposure to reactive oxygen species (ROS) and reactive nitrogen species (RNS) according to US 2014/0275477 A1 as shown in Table 1. The hSCGB1A1 preparations were evaluated with and without LPS (EH100) in Far Western binding experiments with SDC1, SDC4, and GPC3. In this set of blots, the binding of unmodified hSCGB1A1 to HSPGs with or without LPS is barely detectable. The preparations of modified hSCGB1A1 that were treated with NaOC1 (1:15) and peroxynitrite (1:10) showed greater binding to the HSPGs than unmodified, mCPBA (1:10), and NaOC1 (1:1), with and without LPS present, and the strongest binding was to SDC4.

TABLE 1

ROS and RNS-modified hSCGB1A1 preparations

| Prep ID | Chemical modifier | Ratio of protein to oxidizing equivalents |
|---|---|---|
| A | NaOCl | 1:15 |
| B | mCPBA | 1:10 |
| C | Peroxynitrite | 1:10 |
| D | NaOCl | 1:1 |

### Example 10

### SCGBs Bind to Lipids, Phospholipids, Other Lipids

The EMSA method of Example 1 is used to demonstrate binding of SCGB1A1 to one or more selected from among a lipid, phospholipid, surfactant component (dipalmitoyl phosphatidylcholine (DPPC), also known as lecithin, or dipalmitoyl phosphatidylethanolamine), glycerophospholipid, glycolipid, sphingolipid, glycosphingolipid, arachidonic acid, or an eicosanoid, in the absence of added calcium. An alternate method is thin layer chromatography in which spots corresponding to the lipid migrate to different positions in the presence of an SCGB, as described in Mantile et al. (1993). Briefly, photo-activatable lipids and lipid analogues are incubated with SCGBs, then cross-linked by UV exposure. Lipid controls and mixtures containing complexes of SCGBs with lipid moieties are then spotted onto pre-channeled Silica Gel G TLC plates and placed in TLC chambers with petroleum ether/diethyl ether/acetic acid (70:30: 1) eluent. Lipids are stained with iodine vapor and migration of the lipid spots with and without SCGB are compared. This illustrates that one method of synthesizing SCGB preparations of the invention is the use of photo-activatable lipids, lipid analogues, and other compounds to generate covalently linked SCGB complexes that can interact with HSPGs to mediate therapeutic effects.

### Example 11

### Inhibition of Coronavirus Replication

Each CoV attaches to one or more receptors on the surface of infectable cells, thereby gaining entry into susceptible cells that express the receptor(s). The major receptor for SARS-CoV1 and SARS-CoV2 (also known as COVID-19) is the angiotensin converting enzyme (ACE), while the major receptor for MERS-CoV is dipeptidyl peptidase 4 (DPP4) (Tai, 2020), located in membranes of cells of the respiratory tract, digestive tract, and other tissues. The ACE receptor is expressed mainly on the pneumocytes and macrophages in the lungs, which are thought to be the primary cells in humans infected by SARS-CoV2 (Hoffman, 2020). The virus attaches to the ACE receptor via its spike (S) glycoprotein, triggering the cell’s machinery to endocytose the virus and allowing it to enter the cell where it hijacks to make more copies of itself. CC10 has direct antiviral activity against coronaviruses at two levels. The first level of antiviral activity involves the binding of rhCC10 to heparan sulfate side changes of both the coronavirus (CoV) S glycoprotein and its major receptor(s) on cell surfaces, thereby blocking the virus-receptor binding interaction and blocking attachment to the cells surface. The second level of antiviral activity involves the binding interactions between rhCC10 and heparan sulfate proteoglycan proteins (HSPGs). HSPGs, particularly syndecans, play a central role in endocytosis acting as co-receptors for many cell surface proteins via loose interactions between the ligand (or virus) and their glycosaminoglycan side chains that bring the ligand (or virus) closer to the receptor to facilitate receptor binding, as well as formation of endocytic vesicles that aggregate and transport the ligand-receptor pairs, or virus-receptor pairs, into the cell. CC10 binds to HSPGs on the cell surface, blocking the recruitment of virus to the cell surface and facilitating proximity of virus particles to CoV receptors during viral attachment and subsequent endocytosis, thereby disrupting viral transport into the cell.

Methods: The human bronchial epithelial cell line, BEAS-2B, was cultured in RPMI medium containing 10% FBS and antibiotic-antimycotic. Primary human bronchial epithelial cells (HBEpC) obtained from healthy individuals were cultured in Human Bronchial/Tracheal Epithelial Cell Growth Medium containing retinoic acid. Cells were grown in 5% CO₂ at 37° C. BEAS-2B and HBEpC were seeded in 96-well plates and cultured to 90-100% confluence. The media was replaced with fresh media containing coronavirus (CoV) at a multiplicity of infection (MOI) of 3.0, 0.3, or 0 (no virus), with and without rhCC10 at a concentration of 0.5 or 1.5 mg/mL. Virus was incubated with the cells for one hour at 37° C., then the media containing CoV was removed, the cells were washed with PBS, and media containing rhCC10 was added. Cells were incubated at 37° C. for 24, 48, and 72 hours, after which the plates were fixed in 10% formalin to assay for plaques. To decontaminate the plates were kept in formalin for 72 hours, then placed in fresh formalin. Plates were then washed with PBS and blocked with 1% BSA overnight at 4° C. Plates were washed with PBS to remove the blocking solution and incubated in PBS containing Hoechst 33352 nuclear stain at 4° C. After 3-4 hours, the plates were incubated with an anti-CoV antibody that recognizes the CoV spike (S) glycoprotein for 20 minutes on a platform shaker. The plates were then washed in PBS and secondary antibody (goat anti-human AlexaFluor488) was added. After 20 minutes on a platform shaker, the plates were washed and placed in PBS containing the plasma membrane dye (CellMask Deep Red) and left overnight at 4° C. The plates were imaged the next day. S glycoprotein-positive cells were counted and compared to total cell number to determine the percentage of cells infected with the CoV virus.

Results: The percentage of cells infected with CoV is the ratio of the number of CoV S glycoprotein-positive cells to the total number of Hoechst stained nuclei. At 24 hours post-infection, very few cells were infected at either MOI. At 48 hours post-infection, a substantial number of the cells (>20%) were infected at the lowest MOI in the absence of rhCC10. At 48 hours post-infection at the highest MOI, there was a clear dose-dependent reduction in the number of infected cells at both concentrations of rhCC10. At 72 hours post-infection, the reduction in the number of infected cells was significant at both MOI at the highest concentration of rhCC10 (1.5 mg/ml).

### Example 12

### Bacterial Expression and Recovery of rhCC10

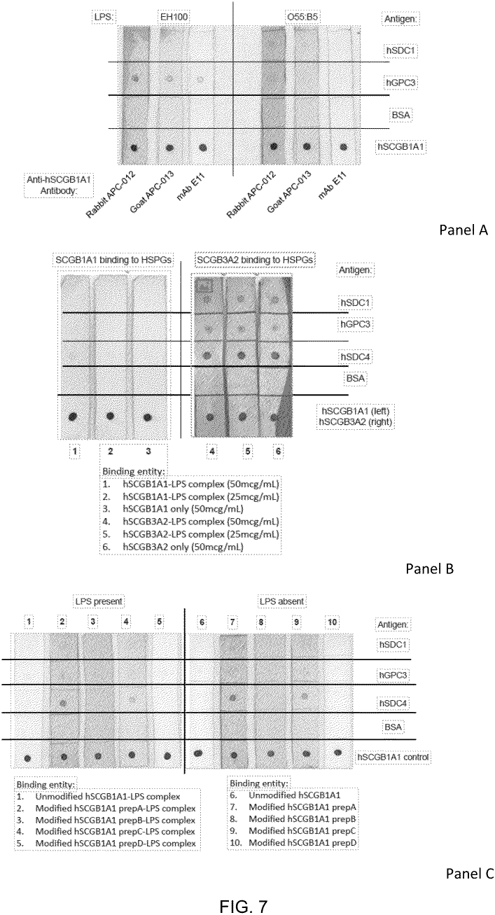

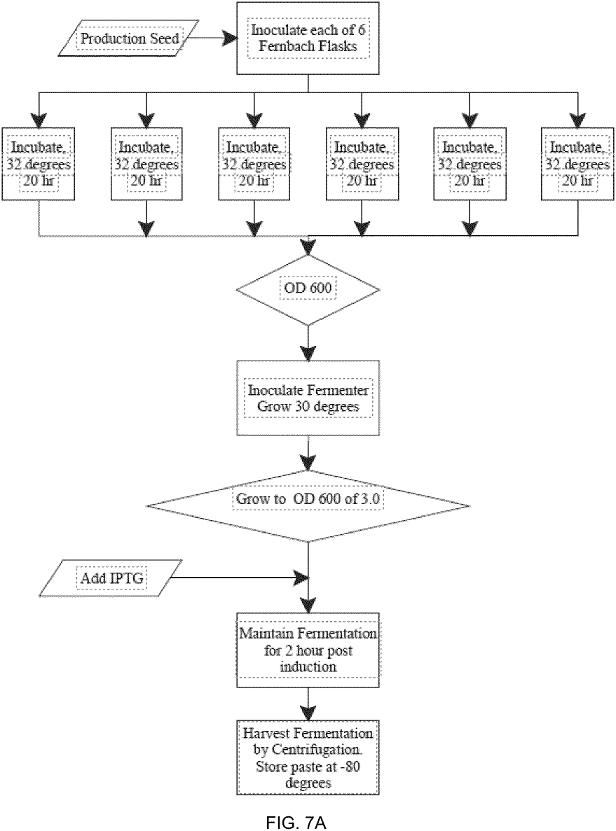

A summary schematic of the bacterial fermentation, expression of rhCC10, and harvest of cell paste containing rhCC10 is shown in FIG. 7A. To begin the fermentation process, a vial of the Production seed cell bank was thawed at room temperature. One hundred microliters of the production seed was then used to inoculate each of the six, fernbach flasks containing one liter each of sterile Super Broth medium (Becton-Dickinson Select APS Super Broth, glycerol and WFI). The cultures in the six flasks were then incubated at 32° C. in a New Brunswick shaker-incubator with agitation (300 rpm) for approximately 20 hours. The cultures in the six flasks were then used to inoculate 300 liters of Superbroth in a 400 liter New Brunswick Scientific Fermenter System (Model IF-400). The culture was grown at 25° C. to 40° C. until a minimum optical density at 600 nm of 2.0 was reached. On reaching a minimum OD₆₀₀ of 2.0 the expression of rhCC10 is induced by the addition of isopropyl-β-Dthiogalactopyranoside (IPTG) to the fermentation culture to a final concentration of 0.1 mM to 10 mM. The fermentation was maintained for at least one hour, preferably two hours post induction. The bacterial culture is harvested by centrifugation with a Sharple’s continuous feed centrifuge. The cell paste is partitioned and stored frozen at -80° C. for later purification.

### Example 13

### Purification of rhCC10

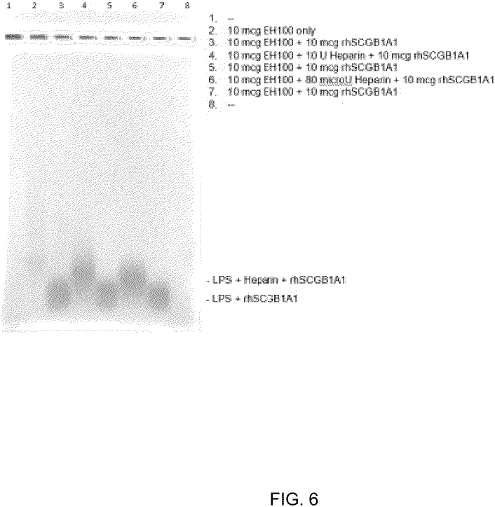

An overview of the rhCC10 purification from bacterial cell paste is shown in FIG. 6. One kilogram of bacterial cell paste was lysed by shear and the cell debris removed by centrifugation. The lysate (supernatant) was then processed using a 100 K nominal molecular weight cut off (NMWCO) membrane in a tangential flow filtration (TFF) system. The permeate from the 100 K step was concentrated by TFF using a 5 K NMWCO membrane and loaded onto a Macro Q anion exchange column. The eluate from the anion exchange column was concentrated and diafiltered by TFF using a 5 K NMWCO membrane before being loaded onto a Type I Hydroxyapatite (HA) column. The eluate from the HA column was then loaded directly onto a column packed with Chelating Sepharose Fast Flow (CSFF) resin with copper as the chelate. The rhCC10 passed through the column while the host-derived proteins present in the HA eluate bound to the column. A positively charged Sartobind Q TFF membrane was also placed into the flowstream after the copper CSFF column to ensure that the maximum amount of endotoxin was removed from the final bulk material. The pass-through from the Sartobind Q was concentrated and then extensively diafiltered using a 5 K NMWCO membrane with saline for injection (SFI) as the replacement buffer, both to remove residual copper as well as to properly formulate the final bulk material.

### Example 14

### Testing of rhCC10

The rhCC10 preparations made by this process, and minor variations thereof, are comparable in all respects: apparent size, molecular weight, charge, N-terminal amino acid sequence, amount of free thiol indicating correct formation of cystine-cystine bonds, immunological recognition techniques such as ELISA and Western blotting, and biological activity. Protein purified using the copper CSFF column was tested for the presence of copper by Inductively Coupled Plasma (by QTI Inc.). No copper was detected and the detection limit of the assay was 0.5 ppm.

The following assays were established as in process assays, characterization assays and release assays for the production process and for the drug substance and drug product. The rhCC10 drug substances and drug products were compared to standard research lot rhCC10/7 where appropriate.

Bacterial Nucleic Acids. Bacterial DNA content per dose of the rhCC10 drug substance and drug product was determined by Southern blot using radiolabeled bacterial DNA followed by hybridization to blotted concentrated rhCC10 sample (Charles River Laboratories-Malvern).

Mass Spectroscopy. The molecular weight was determined by Electrospray Ionization spectrometry by M-Scan Inc. Theoretical molecular weight was determined by PAWS (a shareware program for the determination of average molecular mass, obtained through Swiss Pro). A value of 16110.6 Da was determined by the PAWS program. The same value was found for cGMP batches of rhCC10 and was confirmed by MS analysis of standard research lot rhCC10/7 as a control (determined molecular weight 16110.6 Da).

N-terminal Sequence analysis. The sequence of the N-terminus was carried out using pulsed phase N-terminal sequencing on an Applied Biosystems (ABI) 477A automatic protein sequencer. The analysis was performed by M-Scan Inc. A sequence of Ala-Ala-Glu-Ile was confirmed for cGMP batches of rhCC10 with standard research lot rhCC10/7 as a control.

pH. A three-point calibration (4.0, 7.0, 10.0) is performed according to the manufacturers’ instructions. After calibration of the electrode the pH of the sample is determined.

Isoelectric Focusing. The pI was determined by isoelectric focusing using gels with a pH range of 3 to 7. The gels were obtained from Novex and were run under conditions as described by the manufacturer. Samples were run versus a standard from Sigma and a rhCC10 control (research lot rhCC10/7). Gels were fixed by heating in a microwave for 1 minute in the presence of 10% acetic acid / 30% methanol followed by staining with Gel Code Blue stain from Pierce. Destaining was performed in purified water as described by Pierce.

Free Thiol. The presence of free thiol was determined by reaction with Ellman’s reagent from Pierce using a modified proticol to increase sensitivity. After incubation in the presence of Ellman’s reagent the absorbance of samples was determined in the spectrophotometer at 412 nm. An extinction coefficient of 14150 M-1 cm-1 was used to determine the molar amount of free thiol. A standard curve of free thiol (cysteine) was used to monitor the linearity of the reaction.

LAL. The presence of bacterial endotoxin in rhCC10 process intermediates, drug substance and drug product was tested by the Limulus ameobocyte lysate assay as described in United States Pharmacopeia (USP) Assay No. 85. Kits were obtained from Associates of Cape Cod.

Color, Appearance, Homogeneity. The bulk drug product was visually inspected for clarity, color and visible particulate matter.

Purity and Identity: Reducing SDS PAGE. The rhCC10 drug substance and drug product was run on a Novex 10-20% Tricine SDS-PAGE gel under both reducing and nonreducing conditions as described by the manufacturer. Low molecular weight size standards were obtained from Amersham. Gels were fixed by heating in a microwave for 1 minute in a mixture of 10% acetic acid/ 30% methanol and stained with brilliant blue R250 (0.5%, w/v). Gels were destained with Novex Gel-Clear destaining solution as described by the manufacturer. Gels were then dried using the Novex Gel-Dry system and the percent purity was determined by scanning the gel (Hewlett-Packard scanner Model 5100C) and densitometry was performed using Scion Image shareware from the NIH.

Aggregation Assay. The drug product was analyzed for the presence of aggregates by chromatography on either a Superose 12 or a Sephadex 75 size exclusion chromatography (SEC) column (Amersham/Pharmacia). The column was run according to the manufacturer’s instructions using the BioRad Biologic system and peak area was determined using EZLogic Chromatography Analysis software, also from BioRad. The percent aggregation was determined by comparing the total area of all peaks vs. the area of peaks eluting prior to the main UG peak.

Endotoxin. Endotoxin levels were tested by the rabbit pyrogenicity assay as described in the USP No. 151. An amount of rhCC10 equivalent to a single human dose was administered intravenously over a 10 minute period. Body temperature increase relative to the baseline predose temperature was monitored over the course of three hours. Acceptable results consist of no temperature rise equal to or greater than 0.5° C. over the baseline results.

Protein Content. The protein contents of the process intermediates, drug substance and product were determined by the absorbance at 280 nm using a Shimadzu 120 and an extinction coefficient of 2070 M-1 cm-1 as determined by Mantile et al. (Mantile, 1993).

Sterility. The sterility assay was performed as described in the USP No. 71. Samples were incubated into Fluid Thioglycolate Media (FTM) and Tripticase Soy Broth (TSB). Positive controls for TSB media were C. albicans, A. niger, and B. subtilis. Positive controls for FTM were S. aureus, P. aeruginusa, C. sporogenes.

Testing results for rhCC10 are summarized in Table 2 and wherein positive biological activity test referred to inhibition of PLA₂ activity in U.S. Pat. App. Publication No.: 20030207795, and refers to suppression of viral replication in the present invention.

TABLE 2

Test results for Lot 0728

| | |
|---|---|
| Test | Result |
| Color | Clear, colorless |
| Appearance | No turbidity |
| Homogeneity | Homogeneous |
| Purity | 97.4% |
| Aggregation | 2.25% |
| Sterility | Sterile |
| Biological activity | Positive |
| Bacterial nucleic acid | <1.6 pg/mg |
| Mass spectroscopy | 16112.6 |
| pH | 6.82 |
| Isoelectric focusing | 4.7 |
| Free Thiol | <0.5% (w/w) |
| LAL | <0.01 EU/mg |
| Copper | <16 ❑M |

### Example 15

### Safety and Tolerability of Intranasal Administration of rhCC10

As part of the safety assessment for this proof of concept intranasal administration of rhCC10 in humans adverse events (AEs) and serious adverse events (SAEs) were monitored, recorded and reported. The clinical investigator was responsible for the detection and documentation of events meeting the criteria and definition of an AE or SAE. An AE is any untoward medical occurrence in a subject or a clinical investigation temporally associated with the use of the investigational drug whether or not the event is considered to have a causal relationship with the drug. In this trial, a pre-existing condition (i.e., a disorder present before the AE reporting period started and noted on the pre-treatment medical history/physical examination form) was not reported as an AE unless the condition worsened or episodes increased in frequency during the AE reporting period. Serious adverse events were defined as any untoward medical occurrence that, at any dose; 1) results in death, 2) is life-threatening, 3) requires hospitalization or prolongation of an existing hospitalization, 4) results in disability/incapacity, 5) is a congenital anomaly/birth defect, 6) is an important Other Medical Event (OME), and 7) all grade 4 laboratory abnormalities. The AE reporting period for began upon receiving the first dose of investigational medication and ended at the 2-week post discontinuation of investigational medication visit (follow-up visit).

No SAE’s occurred during the study. Overall, a total of 15 adverse events were reported in subjects in both the placebo and rhCC10 treatment groups. All AEs were rated as mild in severity. In each group, 11 of 15 AEs were rated as non-assessable with respect to relatedness to study drug while four of 15 AEs in each group were rates as unlikely to be related to study drug. A summary of AEs for each patient receiving placebo is given in Table 6 and for those receiving rhCC10 at the time of the AE are given in Table 7.

TABLE 3

List of adverse events for patient receiving placebo

| Patient number | Description | Maximum intensity | Reported as serious? | Relationship to trial drug |
|---|---|---|---|---|
| 6 | Headache | 1 = mild | 0 = No | 1 = unlikely |
| 12 | Gastric influenza | 1 = mild | 0 = No | 4 = not assessable |
| 12 | Gastric influenza | 1 = mild | 0 = No | 4 = not assessable |
| 15 | Ear pain | 1 = mild | 0 = No | 1 = unlikely |
| 15 | headache | 1 = mild | 0 = No | 4 = not assessable |
| 15 | fatigue | 1 = mild | 0 = No | 4 = not assessable |
| 15 | ear pain | 1 = mild | 0 = No | 4 = not assessable |
| 20 | Sore throath | 1 = mild | 0 = No | 4 = not assessable |
| 20 | Common cold | 1 = mild | 0 = No | 4 = not assessable |
| 25 | Headache | 1 = mild | 0 = No | 1 = unlikely |
| 26 | Sore throat | 1 = mild | 0 = No | 4 = not assessable |
| 27 | stomach ache | 1 = mild | 0 = No | 1 = unlikely |
| 29 | common cold | 1 = mild | 0 = No | 4 = not assessable |
| 31 | Fever | 1 = mild | 0 = No | 4 = not assessable |
| 38 | urticaria | 1 = mild | 0 = No | 4 = not assessable |

TABLE 4

List of adverse events for patient receiving rhCC10

| Patient number | Description | Maximum intensity | Reported as serious? | Relationship to trial drug |
|---|---|---|---|---|
| 1 | Common cold | 1 = mild | 0 = No | 4 = not assessable |
| 2 | Common cold | 1 = mild | 0 = No | 1 = unlikely |
| 2 | Common cold | 1 = mild | 0 = No | 1 = unlikely |
| 7 | Sore throat | 1 = mild | 0 = No | 1 = unlikely |
| 16 | fatigue | 1 = mild | 0 = No | 4 = not assessable |
| 16 | fatigue | 1 = mild | 0 = No | 4 = not assessable |
| 23 | Headache | 1 = mild | 0 = No | 4 = not assessable |
| 23 | Common cold | 1 = mild | 0 = No | 4 = not assessable |
| 26 | Common cold | 1 = mild | 0 = No | 4 = not assessable |
| 28 | tired | 1 = mild | 0 = No | 4 = not assessable |
| 28 | tired | 1 = mild | 0 = No | 4 = not assessable |
| 28 | headache | 1 = mild | 0 = No | 4 = not assessable |
| 32 | Headache | 1 = mild | 0 = No | 4 = not assessable |
| 38 | ague | 1 = mild | 0 = No | 4 = not assessable |
| 39 | Mild cold | 1 = mild | 0 = No | 1 = unlikely |

Therefore, intranasal rhCC10 administration was found to be safe and well-tolerated in humans when given once daily as an aerosol in a divided dose of 1.1 milligrams, 0.56 milligrams per nostril, for seven consecutive days.

While it is apparent that the invention herein disclosed is well calculated to fulfill the objects above stated, it will be appreciated that numerous modifications and embodiments may be devised by those skilled in the art. It is intended that the appended claims cover all such modifications and embodiments as fall within the true spirit and scope of the present invention.

We claim:

1. A method of reducing the titer of coronavirus in lung or nasal tissue of a patient comprising; administering human CC10 or rhCC 10 to the patient.

2. The method of claim 1 wherein the rhCC10 treats, cures, or prevents a coronavirus infection in the patient.

3. The method of claim 1 wherein the coronavirus is COVID-19, also known as SARS-CoV2.

4. The method of claim 1 wherein the coronavirus is selected from the group consisting of SARS-CoV1 and MERS-CoV.

5. The method of claim 1 wherein the human CC10 or rhCC10 is administered by the intranasal route.

6. The method of claim 1 wherein the human CC10 or rhCC10 is administered by the intravenous route.

7. The method of claim 1 wherein the human CC10 or rhCC10 is administered by a combination of intranasal and intravenous routes.

8. The method of reducing the titer of a coronavirus in the tissue of a patient comprising; administering human CC10 or rhCC10 to the patient.

9. The method of reducing the titer of a coronavirus in the tissue of a patient comprising; administering a recombinant secretoglobin to the patient.

10. The method of inhibiting viral replication of a coronavirus at the cellular level comprising; administering human CC10 or rhCC10 to the infected patient.

11. The method of inhibiting viral replication of a coronavirus at the cellular level comprising; administering CC10 or recombinant CC10 to the infected animal.

12. The method of inhibiting viral replication of a coronavirus at the cellular level comprising; administering CC10 or recombinant CC10 to the infected cell.
